(19) United States
(12) Patent Application Publication
(10) Pub. No.: US 2018/0128681 A1
(43) Pub. Date: May 10, 2018

(54) IMAGE PROCESSING DEVICE, IMAGING SYSTEM, IMAGE PROCESSING METHOD, AND COMPUTER-READABLE RECORDING MEDIUM

(71) Applicant: OLYMPUS CORPORATION, Tokyo (JP)

(72) Inventor: Takeshi OTSUKA, Tokyo (JP)

(73) Assignee: OLYMPUS CORPORATION, Tokyo (JP)

(21) Appl. No.: 15/862,762

(22) Filed: Jan. 5, 2018

Related U.S. Application Data
(63) Continuation of application No. PCT/JP2015/070330, filed on Jul. 15, 2015.

(51) Int. Cl. G01J 3/28; A61B 5/1455; A61B 1/06

(52) U.S. Cl. CPC G01J 3/2823; A61B 5/14551; G06K 2009/00932; A61B 1/0638; A61B 1/063

(57) ABSTRACT

An image processing device estimates a depth of specified tissue included in an object based on an image obtained by capturing the object with light with wavelengths. The image processing device includes: an absorbance calculating unit configured to calculate absorbances at the wavelengths based on pixel values of pixels constituting the image; a component amount estimating unit configured to estimate each of component amounts by using reference spectra at different depths of tissue for each of two or more kinds of light absorbing components contained respectively in two or more kinds of tissue including the specified tissue based on the absorbances; a ratio calculating unit configured to calculate a ratio of component amounts estimated for a light absorbing component contained in at least the specified tissue; and a depth estimating unit configured to estimate at least a depth of the specified tissue in the object based on the ratio.

### CROSS-REFERENCE TO RELATED APPLICATION

This application is a continuation of International Application No. PCT/JP2015/070330, filed on Jul. 15, 2015, the entire contents of which are incorporated herein by reference.

### BACKGROUND

The present disclosure relates to an image processing device, an imaging system, an image processing method, and a computer-readable recording medium.

One example of physical quantities representing a physical property inherent to an object is a spectral transmittance spectrum. Spectral transmittance is a physical quantity representing a ratio of transmitted light to incident light at each wavelength. While RGB values in an image obtained by capturing an object are information depending on a change in illumination light, camera sensitivity characteristics, and the like, spectral transmittance is information inherent to an object whose value is not changed by exogenous influences. Spectral transmittance is therefore used as information for reproducing original colors of an object in various fields.

Multiband imaging is known as means for obtaining a spectral transmittance spectrum. In multiband imaging, an object is captured by the frame sequential method while 16 bandpass filters, through which illumination light is transmitted, are switched by rotation of a filter wheel, for example. As a result, a multiband image with pixel values of 16 bands at each pixel position is obtained.

Examples of a technique for estimating a spectral transmittance from such a multiband image include an estimation technique using the principal component analysis and an estimation technique using the Wiener estimation. The Wiener estimation is a technique known as one of linear filtering techniques for estimating an original signal from an observed signal with superimposed noise, and minimizes error in the light of the statistical properties of the observed object and the characteristics of noise in observation. Since some noise is contained in a signal from a camera capturing an object, the Wiener estimation is highly useful as a technique for estimating an original signal.

With respect to a point x at a given pixel position in a multiband image, a pixel value g(x,b) in a band b and a spectral transmittance t(x,λ) of light having a wavelength λ at a point on an object corresponding to the point x satisfy the relation of the following formula (1) based on a camera response system.

g(x,b)=∫_{λ}f(b,λ)·s(λ)·e(λ)·t(x,λ)dλ+nₛ(b) (1)

In the formula (1), a function f(b,λ) represents a spectral transmittance of light having the wavelength λ at a b-th bandpass filter, a function s(λ) represents a spectral sensitivity characteristic of a camera at the wavelength λ, a function e(λ) represents a spectral radiation characteristic of illumination at the wavelength λ, and a function nₛ(b) represents observation noise in the band b. Note that a variable b for identifying a bandpass filter is an integer satisfying 1≤b≤16 in the case of 16 bands, for example.

In actual calculation, a relational formula (2) of a matrix obtained by discretizing the wavelength λ is used instead of the formula (1).

G(x)=FSET(x)+N (2)

When the number of sample points in the wavelength direction is represented by m and the number of bands is represented by n, a matrix G(x) is a matrix with n rows and one column having pixel values g(x,b) at points x as elements, a matrix T(x) is a matrix with m rows and one column having spectral transmittances t(x,λ) as elements, and a matrix F is a matrix with n rows and m columns having spectral transmittances f(b,λ) of the filters as elements in the formula (2). In addition, a matrix S is a diagonal matrix with m rows and m columns having spectral sensitivity characteristics s(λ) of the camera as diagonal elements. A matrix E is a diagonal matrix with m rows and m columns having spectral radiation characteristics e(λ) of the illumination as diagonal elements. A matrix N is a matrix with n rows and one column having observation noise nₛ(b) as elements. Note that, since the formula (2) summarizes formulas on a plurality of bands by using matrices, the variable b identifying a bandpass filter is not described. In addition, integration concerning the wavelength λ is replaced by a product of matrices.

Here, for simplicity of description, a matrix H defined by the following formula (3) is introduced. The matrix H is also called a system matrix.

H=FSE (3)

As a result of using the system matrix H, the formula (2) is replaced by the following formula (4).

G(x)=HT(x)+N (4)

For estimation of the spectral transmittance at each point of the object based on a multiband image by using the Wiener estimation, spectral transmittance data T̂(x), which are estimated values of the spectral transmittance, are given by a relational formula (5) of matrices. In the formula, a symbol T̂ means that a symbol “̂ (hat)” representing an estimated value is present over a symbol T. The same applies below.

\hat{T}(x)=WG(x) (5)

A matrix W is called a “Wiener estimation matrix” or an “estimation operator used in the Wiener estimation,” and given by the following formula (6).

W=R_{SS}H^{T}(HR_{SS}H^{T}+R_{NN})⁻¹ (6)

In the formula (6), a matrix R_{SS} is a matrix with m rows and m columns representing an autocorrelation matrix of the spectral transmittance of the object. A matrix R_{NN} is a matrix with n rows and n columns representing an autocorrelation matrix of noise of the camera used for imaging. With respect to a given matrix X, a matrix X^{T} represents a transposed matrix of the matrix X and matrix X⁻¹ represents an inverse matrix of the matrix X. The matrices F, S, and E (see the formula (3)) constituting the system matrix H, that is, the spectral transmittances of the filters, the spectral sensitivity characteristics of the camera, and the spectral radiation characteristics, the matrix R_{SS}, and the matrix R_{NN} are obtained in advance.

Note that, for observation of a thin, translucent object with transmitted light, it is known that dye amounts of an object may be estimated based on the Lambert-Beer law since absorption is dominant in optical phenomena. Hereinafter, a method of observing a stained sliced specimen as an object with a transmission microscope and estimating a dye amount at each point of the object will be explained. More particularly, the dye amounts at points on the object corresponding to respective pixels are estimated based on the spectral transmittance data T̂(x). Specifically, a hematoxylin and eosin (HE) stained object is observed, and estimation is performed on three kinds of dyes, which are hematoxylin, eosin staining cytoplasm, and eosin staining red blood cells or an intrinsic pigment of unstained red blood cells. These names of dyes will hereinafter be abbreviated as a dye H, a dye E, and a dye R. Technically, red blood cells have their own color in an unstained state, and the color of the red blood cells and the color of eosin that has changed during the HE staining process are observed in a superimposed state after the HE staining. Thus, to be exact, combination of the both is referred to as the dye R.

Typically, in a case where the object is a light transmissive material, the intensity I₀(λ) of incident light and the intensity I(λ) of outgoing light at each wavelength λ are known to satisfy the Lambert-Beer law expressed by the following formula (7).

$$\frac{I ( λ )}{{I}_{0} ( λ )} = {e}^{- k ( λ ) \cdot {d}_{0}} \qquad ( 7 )$$

In the formula (7), a symbol k(λ) represents a coefficient unique to a material determined depending on the wavelength λ, and a symbol d₀ represents the thickness of the object.

The left side of the formula (7) means the spectral transmittance t(λ), and the formula (7) is replaced by the following formula (8).

t(λ)=e^{-a(λ)} (8)

In addition, spectral absorbance a(λ) is given by the following formula (9).

a(λ)=k(λ)·d₀ (9)

With the formula (9), the formula (8) is replaced by the following formula (10).

t(λ)=e−a(λ) (10)

When the HE stained object is stained by three kinds of dyes, which are the dye H, the dye E, and the dye R, the following formula (11) is satisfied at each wavelength λ based on the Lambert-Beer law.

$$\frac{I ( λ )}{{I}_{0} ( λ )} = {e}^{- ( {k}_{H} ( λ ) \cdot {d}_{H} + {k}_{E} ( λ ) \cdot {d}_{E} + {k}_{R} ( λ ) \cdot {d}_{R} )} \qquad ( 11 )$$

In the formula (11) coefficients k_{H}(λ), k_{E}(λ), and k_{R}(λ) are coefficients respectively associated with the dye H, the dye E, and the dye R. These coefficients k_{H}(λ), k_{E}(λ), and k_{R}(λ) correspond to dye spectra of the respective dyes staining the object. These dye spectra will hereinafter be referred to as reference dye spectra. Each of the reference dye spectra k_{H}(λ), k_{E}(λ), and k_{R}(λ) may be easily obtained by application of the Lambert-Beer law, by preparing in advance specimens individually stained with the dye H, the dye E, and the dye R and measuring the spectral transmittance of each specimen by a spectroscope.

In addition, symbols d_{H}, d_{E}, and d_{R} are values representing virtual thicknesses of the dye H, the dye E, and the dye R at points of the object respectively corresponding to pixels constituting a multiband image. Since dyes are normally found scattered across an object, the concept of thickness is not correct; however, “thickness” may be used as an index of a relative dye amount indicating how much a dye is present as compared to a case where the object is assumed to be stained with a single dye. In other words, the values d_{H}, d_{E}, and d_{R} may be deemed to represent the dye amounts of the dye H, the dye E, and the dye R, respectively.

When the spectral transmittance at a point of the object corresponding to a point x on the image is represented by t(x,λ), the spectral absorbance is represented by a(x,λ), and the object is stained with three dyes, which are the dye H, the dye E, and the dye R, the formula (9) is replaced by the following formula (12).

a(x,λ)=k(λ)·d_{H}+k_{E}(λ)·d_{E}+k_{R}(λ)·d_{R} (12)

When the estimated spectral transmittance and the estimated absorbance at the wavelength λ of the spectral transmittance T̂(x) are represented by t̂(x,λ) and â(x,λ), respectively, the formula (12) is replaced by the following formula (13).

â(x,λ)=k_{H}(λ)·d_{H}+k_{E}(λ)·d_{E}+k_{R}(λ)·d_{R} (13)

Since unknown variables in the formula (13) are the three dye amounts d_{H}, d_{E}, and d_{R}, the dye amounts d_{H}, d_{E}, and d_{R} may be obtained by creating and calculating simultaneous equations of the formula (13) for at least three different wavelengths λ. In order to increase the accuracy, simultaneous equations of the formula (13) may be created and calculated for four or more different wavelengths λ, so that multiple regression analysis is performed. For example, when three simultaneous equations of the formula (13) for three wavelengths λ₁, λ₂, and λ₃ are created, the equations may be expressed by matrices as the following formula (14).

$$( \begin{matrix}\hat{a} ( x , {λ}_{1} ) \\ \hat{a} ( x , {λ}_{2} ) \\ \hat{a} ( x , {λ}_{3} )\end{matrix} ) = ( \begin{matrix}{k}_{H} ( {λ}_{1} ) & {k}_{E} ( {λ}_{1} ) & {k}_{R} ( {λ}_{1} ) \\ {k}_{H} ( {λ}_{2} ) & {k}_{E} ( {λ}_{2} ) & {k}_{R} ( {λ}_{2} ) \\ {k}_{H} ( {λ}_{3} ) & {k}_{E} ( {λ}_{3} ) & {k}_{R} ( {λ}_{3} )\end{matrix} ) ( \begin{matrix}{d}_{H} \\ {d}_{E} \\ {d}_{R}\end{matrix} ) \qquad ( 14 )$$

The formula (14) is replaced by the following formula (15).

Â(x)=K₀D₀(x) (15)

When the number of samples in the wavelength direction is represented by m, a matrix Â(x) is a matrix with m row and one column corresponding to â(x,λ), a matrix K₀ is a matrix with m rows and three columns corresponding to the reference dye spectra k(λ), and a matrix D₀(x) is a matrix with three rows and one column corresponding to the dye amounts d_{H}, d_{E}, and d_{R} at a point x in the formula (15).

The dye amounts d_{H}, d_{E}, and d_{R} are calculated by using the least squares method according to the formula (15). The least squares method is a method of estimating the matrix D₀(x) such that a sum of squares of error is smallest in a simple linear regression equation. An estimated value D₀̂(x) of the matrix D₀(x) obtained by the least squares method is given by the following formula (16).

\hat{D}₀(x)=(K₀^{T}K₀)⁻¹K₀^{T}Â(x) (16)

In the formula (16), the estimated value D₀̂(x) is a matrix having the estimated dye amounts as elements. Spectral absorbance a˜(x,λ) restored by substituting the estimated dye amounts d̂_{H}, d̂_{E}, and d̂_{R} into the formula (12) is given by the following formula (17). Note that the symbol a˜ means that a symbol “˜(tilde)” representing a restored value is present over a symbol a.

ã(x,λ)=k_{H}(λ)·\hat{d}_{H}+k_{E}(λ)·\hat{d}_{E}+k_{R}(λ)·\hat{d}_{R} (17)

Thus, estimation error e(λ) in the dye amount estimation is given by the following formula (18) from the estimated spectral absorbance â(x,λ) and the restored spectral absorbance a˜(x,λ).

e(λ)=â(x,λ)−ã(x,λ) (18)

The estimation error e(λ) will hereinafter be referred to as a residual spectrum. The estimated spectral absorbance â(x,λ) may be expressed as in the following formula (19) by using the formulas (17) and (18).

â(x,λ)=k_{H}(λ)·\hat{d}_{H}+k_{E}(λ)·\hat{d}_{E}+k_{R}(λ)·\hat{d}_{R}+e(λ) (19)

Note that, although the Lambert-Beer law formulates attenuation of light transmitted by a translucent object on the assumption that no refraction and no scattering occur, refraction and scattering may occur in an actual stained specimen. Thus, modeling of light attenuation caused by the stained specimen with the Lambert-Beer law alone results in an error associated with the modeling. It is, however, highly difficult and practically unfeasible to build a model involving refraction and scattering in a biological specimen. Thus, addition of a residual spectrum, which is an error in modeling in view of the influence of refraction and scattering, prevents occurrence of unnatural color fluctuation due to a physical model.

In observation of reflected light from the object, since the reflected light is affected by optical factors such as scattering in addition to absorption, the Lambert-Beer law may not be applied to the reflected light as it is. In this case, however, setting appropriate constraint conditions allows estimation of the amounts of dye components in the object based on the Lambert-Beer law.

A case of estimation of the amounts of dye components in a fat region near a mucosa of an organ will be described as an example. FIG. 16 is a set of graphs illustrating relative absorbances (reference spectra) of oxygenated hemoglobin, carotene, and bias. Among the graphs, (b) of FIG. 16, illustrates the same data as in (a) of FIG. 16 with a larger scale on the vertical axis and with a smaller range. In addition, bias is a value representing luminance unevenness in an image, which does not depend on the wavelength.

The amounts of respective dye components are calculated from absorption spectra in a region in which fat is imaged based on the reference spectra of oxygenated hemoglobin, carotene, and bias. In this case, the wavelength band is limited to 460 to 580 nm, in which the absorption characteristics of oxygenated hemoglobin contained in blood, which is dominant in a living body, do not change significantly and the wavelength dependence of scattering has little influence, so that optical factors other than absorption do not have influence, and absorbances within the wavelength band are used to estimate the amounts of dye components.

FIG. 17 is a set of graphs illustrating absorbances (estimated values) restored from the estimated amounts of oxygenated hemoglobin according to the formula (14), and measured values of oxygenated hemoglobin. In FIG. 17, (b) shows the same data as in (a) of FIG. 17 with a larger scale on the vertical axis and with a smaller range. As illustrated in FIG. 17, the measured values and the estimated values are approximately the same within the limited wavelength band of 460 to 580 nm. In this manner, even when reflected light from an object is observed, limiting the wavelength band to a narrow range in which the absorption characteristics of the dye components do not significantly change allows estimation of the amounts of components with high accuracy.

In contrast, at the outside of the limited wavelength band, that is, in wavelength bands lower than 460 nm and higher than 580 nm, the measured values and the estimated values are different from one another and estimation error is observed. This is thought to be because the Lambert-Beer law, which expresses absorption phenomena, may not approximate the values since optical factors such as scattering other than absorption affect the reflected light from the object. Thus, it is generally known that the Lambert-Beer law is not satisfied when reflected light is observed.

In recent years, research on measurement of the depth of specified tissue in a living body based on an image capturing the living body has been carried out. For example, JP 2011-098088 A discloses a technology of acquiring a broadband image data corresponding to broadband light in a wavelength band of 470 to 700 nm, for example, and narrow-band image data corresponding to narrow-band light having a wavelength limited to 445 nm, for example, calculating a luminance ratio of pixels at corresponding positions in the broadband image data and the narrow-band image data, obtaining a blood vessel depth corresponding to the calculated luminance ratio based on correlations between luminance ratios and blood vessel depths obtained in advance by experiments or the like, and determining whether or not the blood vessel depth corresponds to a surface layer.

In addition, WO 2013/115323 A discloses a technology of using a difference in optical characteristics between an adipose layer and tissue surrounding the adipose layer at a specified part so as to form an optical image in which a region of an adipose layer including relatively more nerves than surrounding tissue and a region of the surrounding tissue are distinguished from each other, and displaying distribution or a boundary between the adipose layer and the surrounding tissue based on the optical image. This facilitates recognition of the position of the surface of an organ to be removed in an operation to prevent damage to nerves surrounding the organ.

### SUMMARY

An image processing device according to one aspect of the present disclosure is adapted to estimating a depth of specified tissue included in an object based on an image obtained by capturing the object with light with wavelengths, and includes: an absorbance calculating unit configured to calculate absorbances at the wavelengths based on pixel values of pixels constituting the image; a component amount estimating unit configured to estimate each of component amounts by using reference spectra at different depths of tissue for each of two or more kinds of light absorbing components contained respectively in two or more kinds of tissue including the specified tissue based on the absorbances; a ratio calculating unit configured to calculate a ratio of component amounts estimated for a light absorbing component contained in at least the specified tissue; and a depth estimating unit configured to estimate at least a depth of the specified tissue in the object based on the ratio.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a set of graphs illustrating a plurality of reference spectra at different depths of tissue obtained for each of oxygenated hemoglobin and carotene;

FIG. 2 is a set of schematic views illustrating a cross section of a region near a mucosa of a living body;

FIG. 3 is a set of graphs illustrating results of estimation of the amounts of components in a region in which blood is present near a surface of a mucosa;

FIG. 4 is a set of graphs illustrating results of estimation of the amounts of components in a region in which blood is present at a depth;

FIG. 5 is a block diagram illustrating an example configuration of an imaging system according to a first embodiment;

FIG. 6 is a schematic view illustrating an example configuration of an imaging device illustrated in FIG. 5;

FIG. 7 is a flowchart illustrating operation of the image processing device illustrated in FIG. 5;

FIG. 8 is a set of graphs illustrating the estimated amounts of oxygenated hemoglobin;

FIG. 9 is a graph illustrating ratios of the amounts of oxygenated hemoglobin depending on the depth in the region in which blood is present near the surface of the mucosa and in the region in which blood is present at a depth;

FIG. 10 is a block diagram illustrating an example configuration of an image processing device according to a second embodiment;

FIG. 11 is a schematic view illustrating an example of display of a region of fat;

FIG. 12 is a set of graphs for explaining the sensitivity characteristics of an imaging device applicable to the first and second embodiments;

FIG. 13 is a block diagram illustrating an example configuration of an image processing device according to a fourth embodiment;

FIG. 14 is a schematic diagram illustrating an example configuration of an imaging system according to a fifth embodiment;

FIG. 15 is a schematic diagram illustrating an example configuration of an imaging system according to a sixth embodiment;

FIG. 16 is a set of graphs illustrating reference spectra of oxygenated hemoglobin, carotene, and bias in a fat region; and

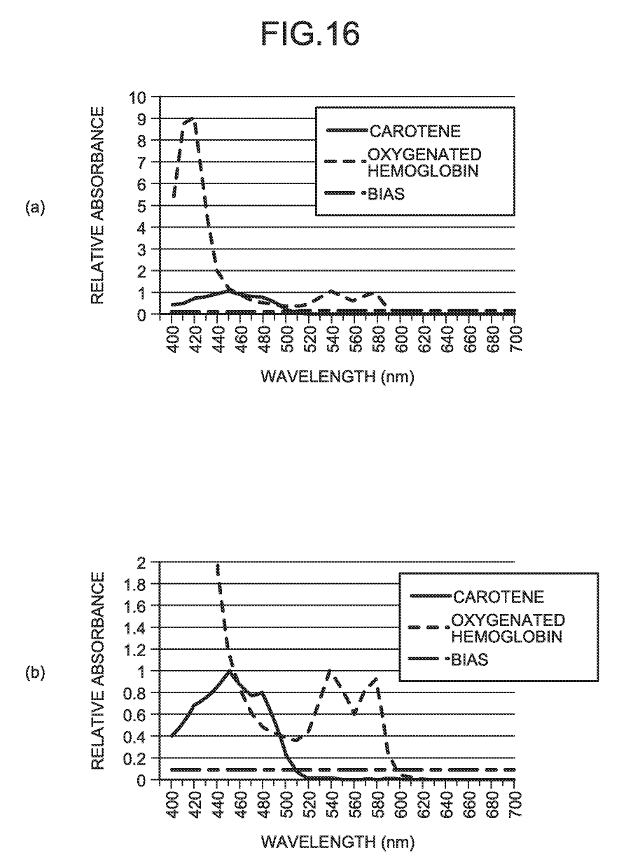

FIG. 17 is a set of graphs illustrating estimated values and measured values of the absorbance of oxygenated hemoglobin.

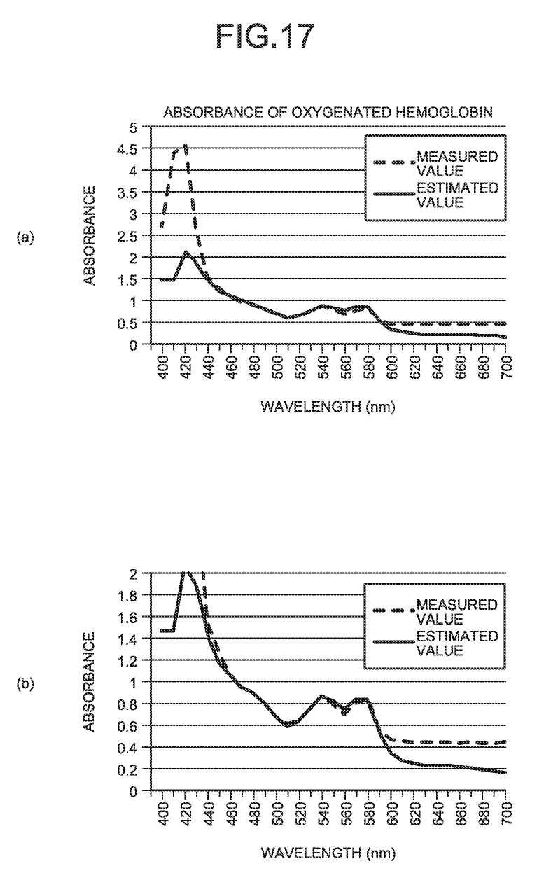

### DETAILED DESCRIPTION

Embodiments of an image processing device, an image processing method, an image processing program, and an imaging system according to the present disclosure will now be described in detail with reference to the drawings. Note that the present disclosure is not limited to the embodiments. In depiction of the drawings, the same components will be designated by the same reference numerals.

### First Embodiment

First, the principle of an image processing method according to a first embodiment will be explained. It is known that a spectrum of a light absorbing component contained in various kinds of tissue present in a living body may not correspond to an absorption spectrum model given by the Lambert-Beer law but may change depending on whether the tissue is at a surface or at a depth. This phenomenon occurs to both of oxygenated hemoglobin, which is a light absorbing component contained in blood, which is major tissue in a living body, and carotene, which is a light absorbing component contained in fat.

It may be attempted to reduce error in estimation of the amount of a component by providing in advance a plurality of reference spectra at different depths of tissue for one kind of light absorbing component by using this phenomenon, and estimating the amount of the component from absorption spectra measured for an object by using the reference spectra having different depths of tissue. Thus, the inventors of the present application have conducted simulation of estimating the amount of a light absorbing component by using reference spectra at different depths of tissue from absorption spectra measured in a wavelength range of 440 to 610 nm for each of oxygenated hemoglobin and carotene.

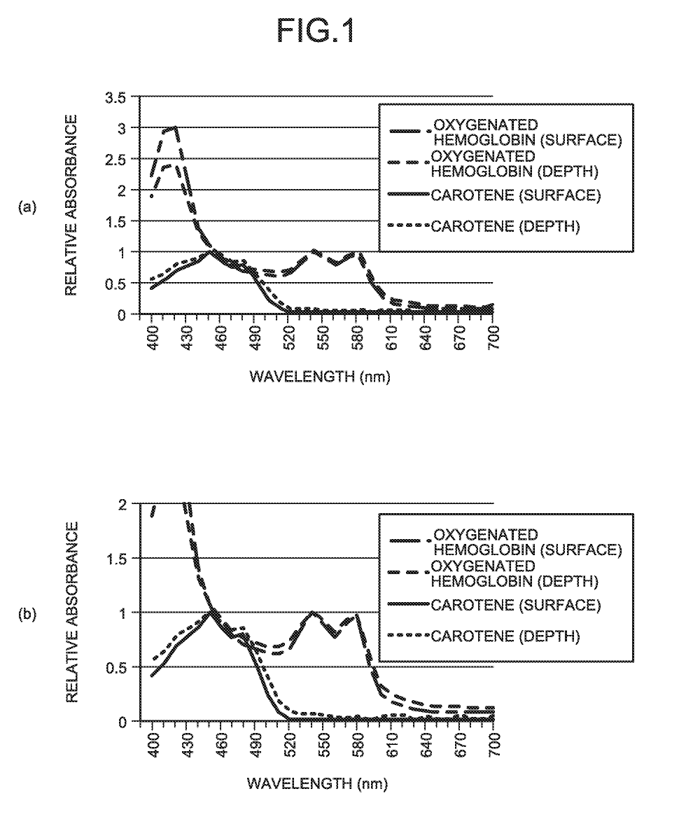

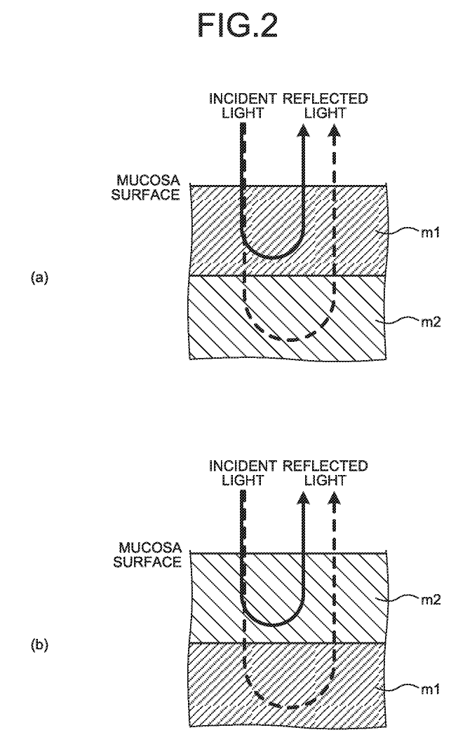

FIG. 1 is a set of graphs illustrating a plurality of reference spectra at different depths of tissue obtained for each of oxygenated hemoglobin and carotene. Among the graphs, (b) of FIG. 1 illustrates the same data as in (a) of FIG. 1 with a larger scale on the vertical axis and with a smaller range. In addition, FIG. 2 is a set of schematic views illustrating a cross section of a region near a mucosa of a living body; Among the schematic views (a) of FIG. 2 illustrates a region in which a blood layer m1 is present near a mucosa surface and a fat layer m2 is present at a depth. (b) of FIG. 2 illustrates a region in which a fat layer m2 is exposed to a mucosa surface and a blood layer m1 is present at a depth.

A graph of oxygenated hemoglobin (surface) illustrated in FIG. 1 represents a reference spectrum of absorbance in a region in which a blood layer m1 is present near a mucosa surface (see (a) of FIG. 2). A graph of oxygenated hemoglobin (depth) represents a reference spectrum of absorbance in a region in which a blood layer m1 is present at a depth and other tissue such as a fat layer m2 is present over the blood layer m1 (see (b) of FIG. 2). A graph of carotene (surface) represents a reference spectrum of absorbance in a region in which a fat layer m2 is exposed to a mucosa surface (see (b) of FIG. 2). A graph of carotene (depth) represents a reference spectrum of absorbance in a region in which a fat layer m2 is present at a depth and other tissue such as blood m1 is present over the fat m2 (see (a) of FIG. 2).

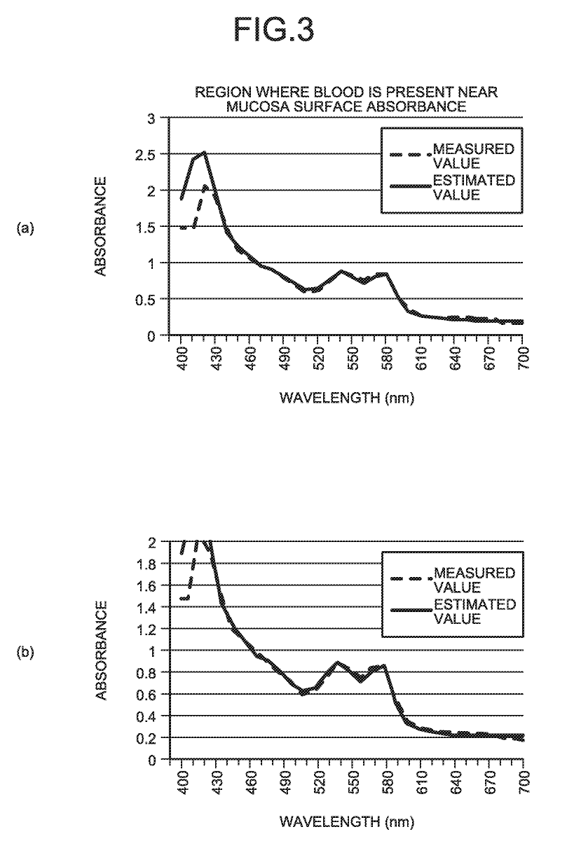

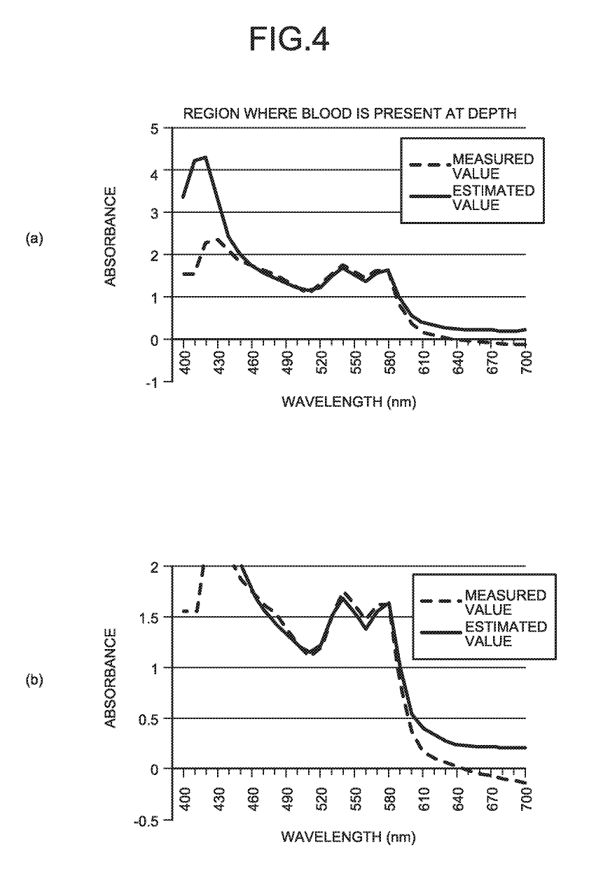

FIG. 3 is a set of graphs illustrating results of estimation of the amounts of components in a region in which blood is present near a surface of a mucosa. In addition, FIG. 4 is a set of graphs illustrating results of estimation of the amounts of components in a region in which blood is present at a depth. Estimated values of absorbance illustrated in FIGS. 3 and 4 are absorbances obtained by estimation of the amounts of respective light absorbing components illustrated in FIG. 1 by using two reference spectra obtained for each of the light absorbing components, and inverse calculation from the estimated amounts of components. Note that a method for estimating the amounts of components will be described later in detail. In FIG. 3, (b) of FIG. 3 illustrates the same data as in (a) of FIG. 3 with a larger scale on the vertical axis and with a smaller range. The same applies to FIG. 4.

As illustrated in FIGS. 3 and 4, it may be seen that computation for estimation of the amounts of components using the reference spectra depending on the depth of tissue allows estimation of the amounts of component with high accuracy in which measured values and estimated values are equal to one another over a wide range from a short wavelength to a long wavelength. In other words, estimation of the amounts of components using a plurality of reference spectra at different depths for one kind of light absorbing component reduces estimation error. Thus, in the first embodiment, the change in estimation error of the amount of component depending on the depth corresponding to a reference spectrum is utilized for estimation of the depth of tissue containing each light absorbing component based on the amount of component estimated by using a plurality of reference spectra at different depths of tissue.

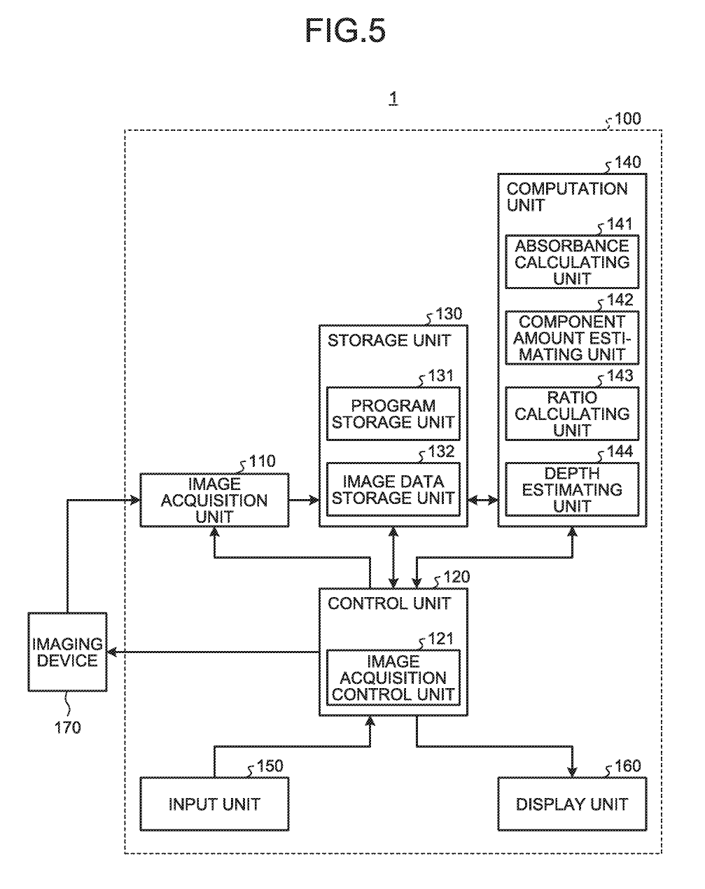

FIG. 5 is a block diagram illustrating an example configuration of an imaging system according to the first embodiment. As illustrated in FIG. 5, the imaging system 1 according to the first embodiment includes an imaging device 170 such as a camera, and an image processing device 100 constituted by a computer such as a personal computer connectable with the imaging device 170.

The image processing device 100 includes an image acquisition unit 110 for acquiring image data from the imaging device 170, a control unit 120 for controlling overall operation of the system including the image processing device 100 and the imaging device 170, a storage unit 130 for storing image data and the like acquired by the image acquisition unit 110, a computation unit 140 for performing predetermined image processing based on the image data stored in the storage unit 130, an input unit 150, and a display unit 160.

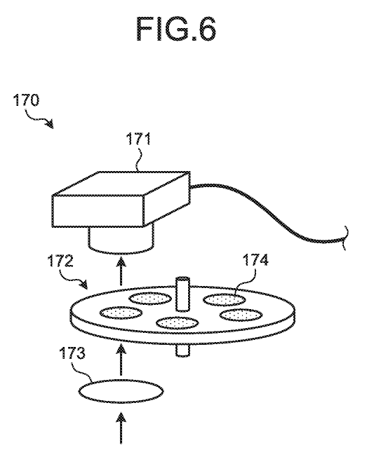

FIG. 6 is a schematic view illustrating an example configuration of the imaging device 170 illustrated in FIG. 5. The imaging device 170 illustrated in FIG. 6 includes a monochromatic camera 171 for generating image data by converting received light into electrical signals, a filter unit 172, and a tube lens 173. The filter unit 172 includes a plurality of optical filters 174 having different spectral characteristics, and switches between optical filters 174 arranged on an optical path of light incident on the monochromatic camera 171 by rotating a wheel. For capturing a multiband image, the optical filters 174 having different spectral characteristics are sequentially positioned on the optical path, and an operation of causing reflected light from an object to form an image on a light receiving surface of the monochromatic camera 171 via the tube lens 173 and the filter unit 172 is repeated for each of the filters 174. Note that the filter unit 172 may be provided on the side of an illumination device for irradiating an object instead of the side of the monochromatic camera 171.

In addition, a multiband image may be acquired in such a manner that an object is irradiated with light having different wavelengths in respective bands. In addition, the number of bands of a multiband image is not limited as long as the number is not smaller than the number of kinds of light absorbing components contained in an object. For example, the number of bands may be three such that an RGB image is acquired.

Alternatively, liquid crystal tunable filter or an acousto-optic tunable filter capable of changing spectral characteristics may be used instead of the optical filters 174 having different spectral characteristics. In addition, a multiband image may be acquired in such a manner that a plurality of light beams having different spectral characteristics may be switched to irradiate an object.

With reference back to FIG. 5, the image acquisition unit 110 has an appropriate configuration depending on the mode of the system including the image processing device 100. For example, in a case where the imaging device 170 illustrated in FIG. 5 is connected to the image processing device 100, the image acquisition unit 110 is constituted by an interface for reading image data output from the imaging device 170. Alternatively, in a case where a server for saving image data generated by the imaging device 170 is provided, the image acquisition unit 110 is constituted by a communication device or the like connected with the server, and acquires image data through data communication with the server. Alternatively, the image acquisition unit 110 may be constituted by a reader, on which a portable recording medium is removably mounted, for reading out image data recorded on the recording medium.

The control unit 120 is constituted by a general-purpose processor such as a central processing unit (CPU) or a special-purpose processor such as various computation circuits configured to perform specific functions such as an application specific integrated circuit (ASIC). In a case where the control unit 120 is a general-purpose processor, the control unit 120 performs providing instructions, transferring data, and the like to respective components of the image processing device 100 by reading various programs stored in the storage unit 130 to generally control the overall operation of the image processing device 100. In a case where the control unit 120 is a special-purpose processor, the processor may perform various processes alone or may use various data and the like stored in the storage unit 130 so that the processor and the storage unit 130 perform various processes in cooperation or in combination.

The control unit 120 includes an image acquisition control unit 121 for controlling operation of the image acquisition unit 110 and the imaging device 170 to acquire an image, and controls the operation of the image acquisition unit 110 and the imaging device 170 based on an input signal input from the input unit 150, an image input from the image acquisition unit 110, and a program, data, and the like stored in the storage unit 130.

The storage unit 130 is constituted by various IC memories such as a read only memory (ROM) or a random access memory (RAM) such as an updatable flash memory, an information storage device such as a hard disk or a CD-ROM that is built in or connected via a data communication terminal, a writing/reading device that reads/writes information from/to the information storage device, and the like. The storage unit 130 includes a program storage unit 131 for storing image processing programs, and an image data storage unit 132 for storing image data, various parameters, and the like to be used during execution of the image processing programs.

The computation unit 140 is constituted by a general-purpose processor such as a CPU or a special-purpose processor such as various computation circuits for performing specific functions such as an ASIC. In a case where the computation unit 140 is a general-purpose processor, the processor reads an image processing program stored in the program storage unit 131 so as to perform image processing of estimating a depth at which specified tissue is present based on a multiband image. Alternatively, in a case where the computation unit 140 is a special-purpose processor, the processor may perform various processes alone or may use various data and the like stored in the storage unit 130 so that the processor and the storage unit 130 perform image processing in cooperation or in combination.

More specifically, the computation unit 140 includes an absorbance calculating unit 141, a component amount estimating unit 142, a ratio calculating unit 143, and a depth estimating unit 144. The absorbance calculating unit 141 calculates absorbance in an object based on an image acquired by the image acquisition unit 110. The component amount estimating unit 142 estimates the amounts of a plurality of components by using a plurality of reference spectra at different depths of tissue for each of light absorbing components respectively contained in a plurality of kinds of tissue present in the object. The ratio calculating unit 143 calculates a ratio of the amounts of each of the light absorbing components at different depths. The depth estimating unit 144 estimates the depth of tissue containing a light absorbing component based on the ratio of the amounts of components calculated for each of the light absorbing components.

The input unit 150 is constituted by input devices such as a keyboard, a mouse, a touch panel, and various switches, for example, and outputs, to the control unit 120, input signals in response to operational inputs.

The display unit 160 is constituted by a display device such as a liquid crystal display (LCD), an electroluminescence (EL) display, or a cathode ray tube (CRT) display, and displays various screens based on display signals input from the control unit 120.

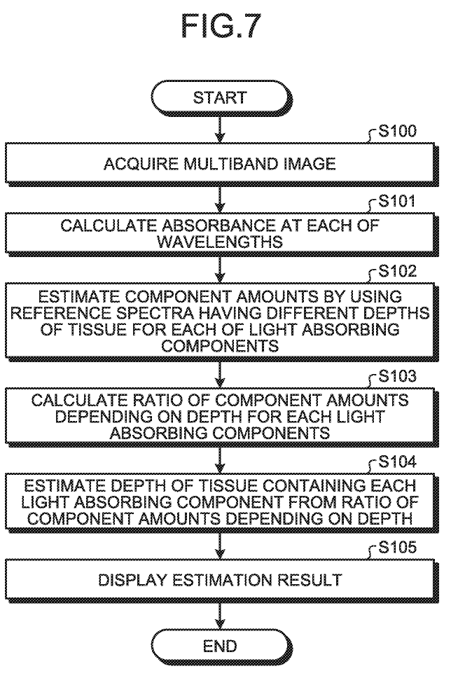

FIG. 7 is a flowchart illustrating operation of the image processing device 100. First, in step S100, the image processing device 100 causes the imaging device 170 to operate under the control of the image acquisition control unit 121 to acquire a multiband image obtained by capturing an object with light with plurality of wavelengths. In the first embodiment, multiband imaging in which the wavelength is sequentially shifted by 10 nm between 400 and 700 nm is performed. The image acquisition unit 110 acquire image data of the multiband image generated by the imaging device 170, and stores the image data in the image data storage unit 132. The computation unit 140 acquires the multiband image by reading the image data from the image data storage unit 132.

In subsequent step S101, the absorbance calculating unit 141 obtains pixel values of a plurality of pixels constituting the multiband image, and calculates absorbance at each of the wavelengths based on the pixel values. Specifically, the value of a logarithm of a pixel value in a band corresponding to each wavelength λ is assumed to be an absorbance a(λ) at the wavelength. Hereinafter, a matrix with m rows and one column having absorbances a(λ) at m wavelengths λ as elements is represented by an absorbance matrix A.

In subsequent step S102, the component amount estimating unit 142 estimates the amounts of a plurality of components by using a plurality of reference spectra at different depths of tissue for each of light absorbing components present respectively in a plurality of kinds of tissue of the object. Hereinafter, a case in which two kinds of light absorbing components, which are oxygenated hemoglobin and carotene, are present in an object, and the amounts of components are calculated by using two reference spectra of depths for each of the light absorbing components. The reference spectra at different depths of tissue are acquired and stored in the storage unit 130 in advance.

A reference spectrum with a deep depth and a reference spectrum with a shallow depth, which are acquired in advance for oxygenated hemoglobin, are represented by k₁₁(λ) and k₁₂(λ), respectively. A reference spectrum with a shallow depth and a reference spectrum with a deep depth, which are acquired in advance for carotene, are represented by k₂₁(λ) and k₂₂(λ), respectively. In addition, the amount of oxygenated hemoglobin calculated based on the reference spectrum k₁₁(λ) is represented by d₁₁, the amount of oxygenated hemoglobin calculated based on the reference spectrum k₁₂(λ) is represented by d₁₂, the amount of carotene calculated based on the reference spectrum k₂₁(λ) is represented by d₂₁, and the amount of carotene calculated based on the reference spectrum k₂₂(λ) is represented by d₂₂.

The relation of the following formula (20) is satisfied between the reference spectra k₁₁(λ), k₁₂(λ), k₂₁(λ), and k₂₂ (λ) and the component amounts d₁₁, d₁₂, d₂₁, and d₂₂, and the absorbance a(λ).

a(λ)=k₁₁(λ)·d₁₁+k₁₂(λ)·d₁₂+k₂₁(λ)·d₂₁+k₂₂(λ)·d₂₂+k_{bias}(λ)·d_{bias} (20)

In the formula (20), the bias d_{bias} is a value representing luminance unevenness in an image, which does not depend on the wavelength. Hereinafter, the bias d_{bias} is treated similarly to the component amounts in computation.

In the formula (20), since there are five unknown variables, which are d₁₁, d₁₂, d₂₁, d₂₂, and d_{bias}, these variables may be solved by calculating simultaneous equations of the formula (20) for at least five different wavelengths X. In order to increase the accuracy, simultaneous equations of the formula (20) may be calculated for five or more different wavelengths λ, so that multiple regression analysis is performed. For example, when simultaneous equations of the formula (20) for five wavelengths λ₁, λ₂, λ₃, λ₄ and λ₅ are created, the equations may be expressed by matrices as the following formula (21).

$$( \begin{matrix}a ( {λ}_{1} ) \\ a ( {λ}_{2} ) \\ a ( {λ}_{3} ) \\ a ( {λ}_{4} ) \\ a ( {λ}_{5} )\end{matrix} ) = ( \begin{matrix}{k}_{11} ( {λ}_{1} ) & {k}_{12} ( {λ}_{1} ) & {k}_{21} ( {λ}_{1} ) & {k}_{22} ( {λ}_{1} ) & {k}_{bias} ( {λ}_{1} ) \\ {k}_{11} ( {λ}_{2} ) & {k}_{12} ( {λ}_{2} ) & {k}_{21} ( {λ}_{2} ) & {k}_{22} ( {λ}_{2} ) & {k}_{bias} ( {λ}_{2} ) \\ {k}_{11} ( {λ}_{3} ) & {k}_{12} ( {λ}_{3} ) & {k}_{21} ( {λ}_{3} ) & {k}_{22} ( {λ}_{3} ) & {k}_{bias} ( {λ}_{3} ) \\ {k}_{11} ( {λ}_{4} ) & {k}_{12} ( {λ}_{4} ) & {k}_{21} ( {λ}_{4} ) & {k}_{22} ( {λ}_{4} ) & {k}_{bias} ( {λ}_{4} ) \\ {k}_{11} ( {λ}_{5} ) & {k}_{12} ( {λ}_{5} ) & {k}_{21} ( {λ}_{5} ) & {k}_{22} ( {λ}_{5} ) & {k}_{bias} ( {λ}_{5} )\end{matrix} ) ( \begin{matrix}{d}_{11} \\ {d}_{12} \\ {d}_{21} \\ {d}_{22} \\ {d}_{bias}\end{matrix} ) \qquad ( 21 )$$

Furthermore, the formula (21) is replaced by the following formula (22).

A=KD (22)

In the formula (22), a matrix A is a matrix with m rows and one column having absorbances at m wavelengths λ (m=5 in the formula (21)) as elements. A matrix K is a matrix with m rows and five columns having, as elements, values of a plurality of kinds of reference spectra at the wavelengths λ acquired for each of the light absorbing component. A matrix D is a matrix with m rows and one column having unknown variables (component amounts) as elements.

The formula (22) is solved by the least squares method to calculate the component amounts d₁₁, d₁₂, d₂₁, d₂₂, and d_{bias}. The least squares method is a method of determining d₁₁, d₁₂, . . . such that a sum of squares of error is smallest in a simple linear regression equation, and is solved by the following formula (23).

D=(K^{T}K)⁻¹K^{T}A (23)

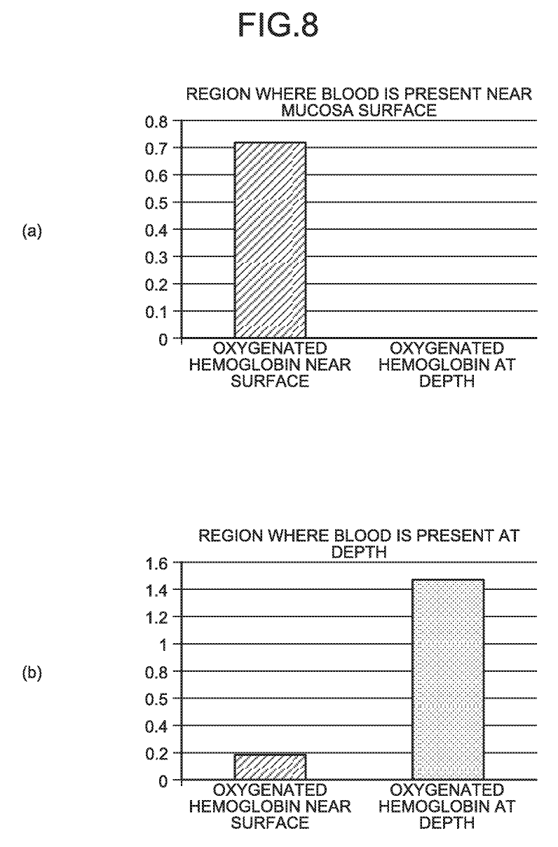

FIG. 8 is a set of graphs illustrating the estimated amount of oxygenated hemoglobin. Among the graphs, (a) of FIG. 8 illustrates the amount of oxygenated hemoglobin in a region in which blood is present near a mucosa surface, and (b) of FIG. 8 illustrates the amount of oxygenated hemoglobin contained in a region in which blood is present at a depth.

As illustrated in (a) of FIG. 8, in a region in which blood is present near the surface, the amount of hemoglobin d₁₁ at a shallow depth is overwhelmingly large and the amount of hemoglobin d₁₂ at a shallow depth is very small. In contrast, as illustrated in (b) of FIG. 8, in a region in which blood is present at a depth, the amount of hemoglobin d₁₁ at a shallow depth is smaller than the amount of hemoglobin d₁₂ at a deep depth.

In subsequent step S103, the ratio calculating unit 143 calculates a ratio of the amounts of each of the light absorbing components depending on the depth. Specifically, a ratio drate₁ of the component amount d₁₁ near the surface to a sum d₁₁+d₁₂ of the amount of oxygenated hemoglobin from the vicinity of the surface to the depth is calculated by a formula (24-1). In addition, a ratio drate₂ of the component amount d₂₁ near the surface to a sum d₂₁+d₂₂ of the amount of carotene from the vicinity of the surface to the depth is calculated by a formula (24-2).

$$\begin{matrix}{drate}_{1} = \frac{{d}_{11}}{{d}_{11} + {d}_{12}} & ( 24 \text{-} 1 ) \\ {drate}_{2} = \frac{{d}_{21}}{{d}_{21} + {d}_{22}} & ( 24 \text{-} 2 )\end{matrix}$$

In subsequent step S104, the depth estimating unit 144 estimates the depth of tissue containing each light absorbing component from the ratio of the component amounts depending on the depth. More specifically, the depth estimating unit 144 first calculates evaluation functions E_{drate1} and E_{drate2} by the following formulas (25-1) and (25-2), respectively.

E_{drate1}=drate₁−T_{drate1} (25-1)

E_{drate2}=drate₂−T_{drate2} (25-2)

The evaluation function E_{drate1} given by the formula (25-1) is for determination on whether the depth of blood containing oxygenated hemoglobin is shallow or deep. A threshold T_{drate1} in the formula (25-1) is a fixed value of 0.5 or the like or a value determined based on experiments or the like set in advance and stored in the storage unit 130. In addition, the evaluation function E_{drate2} given by the formula (25-2) is for determination on whether the depth of fat containing carotene is shallow or deep. A threshold T_{drate2} in the formula (25-2) is a fixed value of 0.9 or the like or a value determined based on experiments or the like set in advance and stored in the storage unit 130.

The depth estimating unit 144 determines that blood is present near the surface of a mucosa when the evaluation function E_{drate1} is zero or positive, that is when the ratio drate₁ of the depth is not smaller than the threshold T_{drate1}, and determines that blood is present at a depth when the evaluation function E_{drate1} is negative, that is when the ratio drate₁ is smaller than the threshold T_{drate1}.

In addition, the depth estimating unit 144 determines that fat is present near the surface of a mucosa when the evaluation function E_{drate2} is zero or positive, that is when the ratio drate₂ of the depth is not smaller than the threshold T_{drate2}, and determines that fat is present at a depth when the evaluation function E_{drate2} is negative, that is when the ratio drate₂ is smaller than the threshold T_{drate2}.

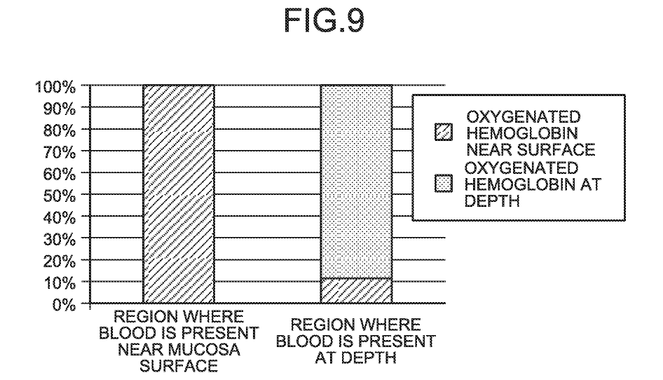

FIG. 9 is a graph illustrating ratios of the amounts of oxygenated hemoglobin depending on the depth in the region in which blood is present near the surface of the mucosa and in the region in which blood is present at a depth. As illustrated in FIG. 9, the amount of oxygenated hemoglobin near the surface occupies a greater part in the region in which blood is present near the mucosa surface. In contrast, the amount of oxygenated hemoglobin at a depth occupies a greater part in the region in which blood is present at a depth.

In subsequent step S105, the computation unit 140 outputs an estimation result, and the control unit 120 displays the estimation result on the display unit 160. The mode in which the estimation result is displayed is not particularly limited. For example, different false colors or hatching of different patterns may be applied on the region in which blood is estimated to be present near the surface of the mucosa and the region in which blood is estimated to be present at a depth, and displayed on the display unit 160. Alternatively, contour lines of different colors may be superimposed on these regions. Furthermore, highlighting may be applied in such a manner that the luminance of a false color or hatching may be increased or caused to blink so that either one of these region is more conspicuous than the other.

As described above, according to the first embodiment, a plurality of component amounts are calculated for each light absorbing component by using a plurality of reference spectra at different depths, and the depth of tissue containing the light absorbing component is estimated based on the ratio of the component amounts, which allows estimation of the depth of tissue with high accuracy even when a plurality of kinds of tissue containing different light absorbing components are present in an object.

While the depth of blood is estimated through estimation of the amounts of two kinds of light absorbing components respectively contained in two kinds of tissue, which are blood and fat, in the first embodiment described above, three or more kinds of light absorbing components may be used. For example, for skin analysis, the amounts of three kinds of light absorbing components, which are hemoglobin, melanin, and bilirubin, contained in tissue near the skin may be estimated. Note that hemoglobin and melanin are major pigments constituting the color of the skin, and bilirubin is a pigment appearing as a symptom of jaundice.

### Modification

The method for estimating the depth performed by the depth estimating unit 144 is not limited to the method explained in the first embodiment. For example, a table or a formula associating the value of the ratios drate₁, drate₂ of the component amounts depending on the depth with the depths may be provided in advance and a specific depth may be obtained based on the table or formula.

Alternatively, the depth estimating unit 144 may estimate the depth of blood based on the ratio drate₁ of the component amounts depending on the depth calculated for hemoglobin. Specifically, the depth of blood is determined to be shallower as the ratio drate₁ is larger, and determined to be deeper as the ratio drate₁ is larger.

Alternatively, the ratio of the amount d₁₂ of oxygenated hemoglobin at a depth to a sum d₁₁+d₁₂ of the amounts of oxygenated hemoglobin from the vicinity of the surface to a depth may be calculated as the ratio of the component amounts, and in this case, the depth estimating unit 144 determines that the depth of blood is deeper as the ratio is larger. Alternatively, the blood may be determined to be at a depth when the ratio is not smaller than a threshold, and determined to be near a mucosa surface when the ratio is smaller than the threshold.

### Second Embodiment

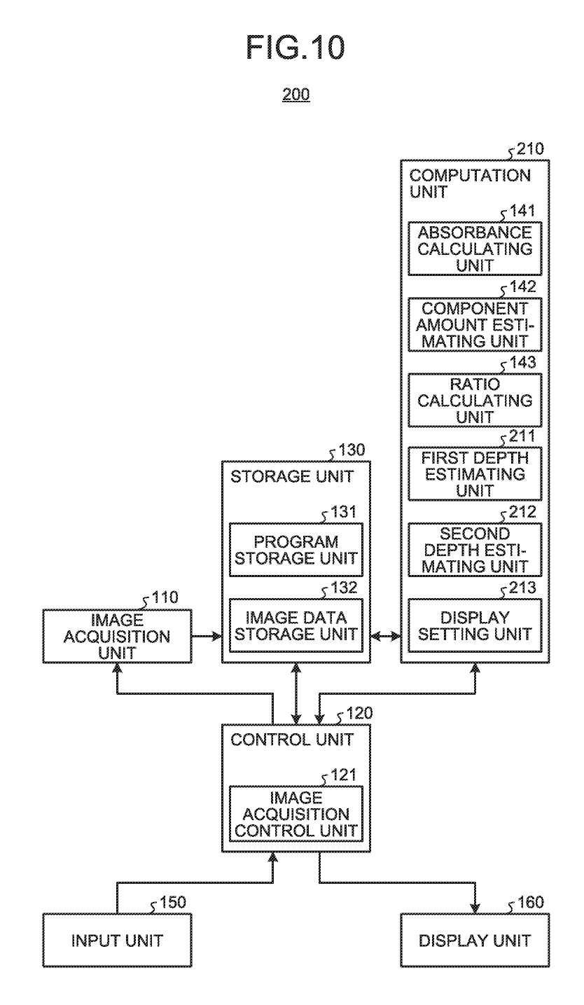

Next, a second embodiment will be described. FIG. 10 is a block diagram illustrating an example configuration of an image processing device according to the second embodiment. As illustrated in FIG. 10, an image processing device 200 according to the second embodiment includes a computation unit 210 instead of the computation unit 140 illustrated in FIG. 4. The configurations and the operations of the respective components of the image processing device 200 other than the computation unit 210 are similar to those in the first embodiment. In addition, the configuration of an imaging device from which the image processing device 200 acquires an image is also similar to that in the first embodiment.

Note that fat observed in a living body includes fat exposed to the surface of a mucosa (exposed fat) and fat that is covered by a mucosa and may be seen therethrough (submucosal fat). In terms of operative procedures, submucosal fat is important. This is because exposed fat may be easily seen with eyes. Thus, technologies for such display that allows operators to easily recognize submucosal fat have been desired. In the second embodiment, the depth of fat is estimated based on the depth of blood, which is major tissue in a living body so that recognition of the submucosal fat is facilitated.

The computation unit 210 includes a first depth estimating unit 211, a second depth estimating unit 212, and a display setting unit 213 instead of the depth estimating unit 144 illustrated in FIG. 5. The operations of the absorbance calculating unit 141, the component amount estimating unit 142, and the ratio calculating unit 143 are similar to those in the first embodiment.

The first depth estimating unit 211 estimates the depth of blood based on the ratio of the amounts of hemoglobin at different depths calculated by the ratio calculating unit 143. The method for estimating the depth of blood is similar to that in the first embodiment (see step S103 in FIG. 7).

The second depth estimating unit 212 estimates the depth of tissue other than blood, that is specifically fat, based on the result of estimation by the first depth estimating unit 211. Note that two or more kinds of tissue have a layered structure in a living body. For example, a mucosa in a living body has a region in which a blood layer m1 is present near the surface and a fat layer m2 is present at a depth as illustrated in (a) of FIG. 2, or a region in which a fat layer m2 is present near the surface and a blood layer m1 is present at a depth as illustrated in (b) of FIG. 2.

Thus, when a blood layer m1 is estimated to be present near the surface by the first depth estimating unit 211, the second depth estimating unit 212 estimates that a fat layer m2 is present at a depth. Conversely, when a blood layer m1 is estimated to be present at a depth by the first depth estimating unit 211, the second depth estimating unit 212 estimates that a fat layer m2 is present near the surface. Estimation of the depth of blood, which is major tissue in a living body, in this manner allows estimation of the depth of other tissue such as fat.

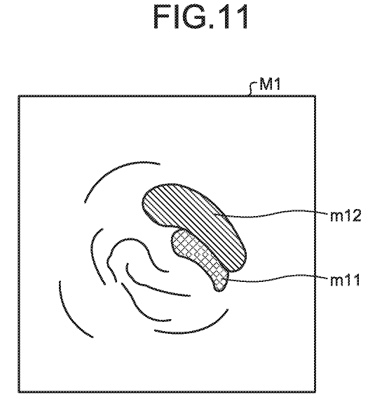

The display setting unit 213 sets a display mode of a region of fat in an image to be displayed on the display unit 160 according to the depth estimation result from the second depth estimating unit 212. FIG. 11 is a schematic view illustrating an example of display of a region of fat. As illustrated in FIG. 11, the display setting unit 213 sets, in an image M1, different display modes for a region m11 in which blood is estimated to be present near the surface of a mucosa and fat is estimated to be present at a depth and a region m12 in which blood is estimated to be present at a depth and fat is estimated to be present near the surface. In this case, the control unit 120 displays the image M1 on the display unit 160 according to the display mode set by the display setting unit 213.

Specifically, when false colors or hatching is applied to all the regions in which fat is present, different colors or patterns are used for the region m11 in which fat is present at a depth and the region m12 in which fat is exposed to the surface. Alternatively, only either one of the region m11 in which fat is present at a depth and the region m12 in which fat is exposed to the surface may be colored. Still alternatively, the signal value of an image signal for display may be adjusted so that the false color changes depending on the amount of fat instead of uniform application of a false color.

In addition, contour lines of different colors may be superimposed on the regions m11 and m12. Furthermore, highlighting may be applied in such a manner that the false color or the contour line in either of the regions m11 and m12 is caused to blink or the like.

Such a display mode of the regions m11 and m12 may be appropriately set according to the purpose of observation. For example, in a case where an operation to remove an organ such as a prostate, there is a demand for facilitating recognition of the position of fat in which many nerves are present. Thus, in this case, the region m11 in which the fat layer m2 is present at a depth is preferably displayed in a more highlighted manner.

As described above, according to the second embodiment, since the depth of blood, which is major tissue in a living body, is estimated and the depth of other tissue such as fat is estimated based on the relation with the major tissue, the depth of tissue other than major tissue may also be estimated in a region in which two or more kinds of tissue are layered.

In addition, according to the second embodiment, since the display mode in which the regions are displayed is changed depending on the positional relation of blood and fat, a viewer of the image is capable of recognize the depth of tissue of interest more clearly.

### Third Embodiment

Next, a third embodiment will be described. While it is assumed that multispectral imaging is performed in the first and second embodiments described above, imaging with any three wavelengths is sufficient for estimation of three values, which are the amounts of two components and the bias.

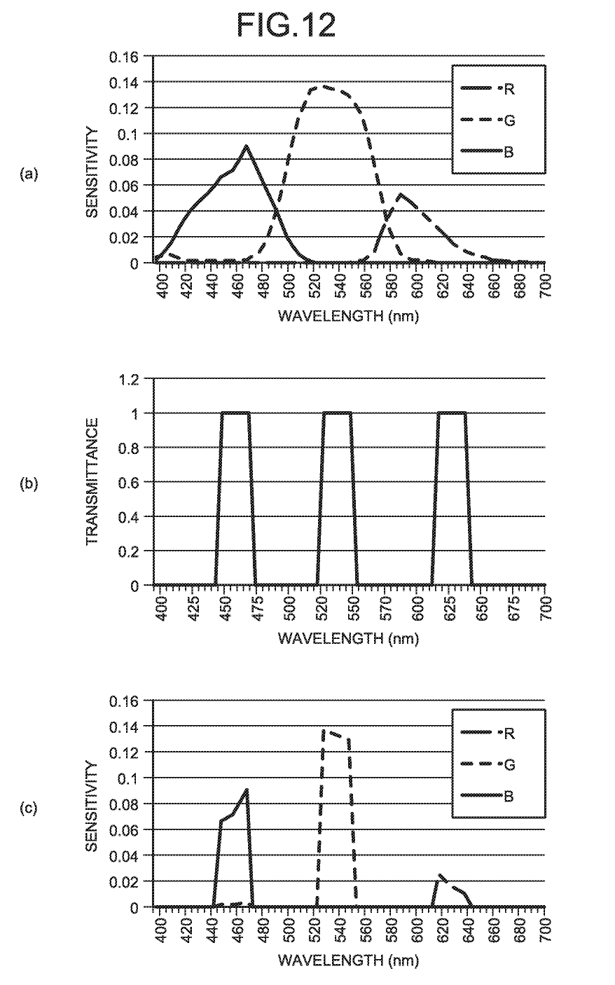

In this case, the imaging device 170 from which the image processing devices 100 and 200 obtain an image may have a configuration including an RGB camera with a narrow-band filter. FIG. 12 is a set of graphs for explaining the sensitivity characteristics of such an imaging device. (a) of FIG. 12 illustrates the sensitivity characteristics of an RGB camera, (b) of FIG. 12 illustrates the transmittance of a narrow-band filter, and (c) of FIG. 12 illustrates the total sensitivity characteristics of the imaging device.

When an object image is formed with RGB via the narrow-band filter, the total sensitivity characteristics of the imaging device are given by a product (see (c) of FIG. 12) of the sensitivity characteristics of the camera (see (a) of FIG. 12) and the sensitivity characteristics of the narrow-band filter (see (b) of FIG. 12).

### Fourth Embodiment

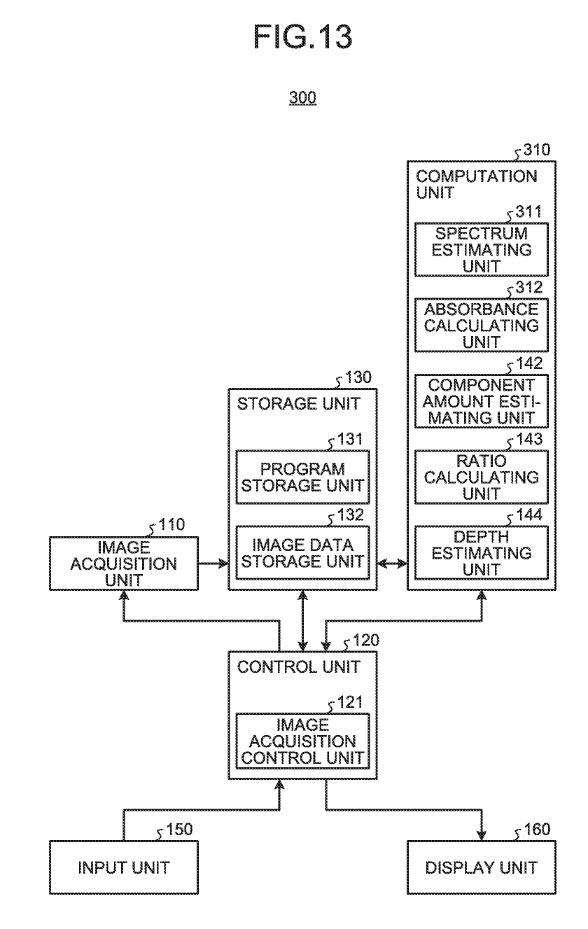

Next, a fourth embodiment is described. While it is assumed that multispectral imaging is performed in the first and second embodiments, an optical spectrum may be estimated with use of a small number of bands, and the amount of a light absorbing component may be estimated from the estimated optical spectrum. FIG. 13 is a block diagram illustrating an example configuration of an image processing device according to the fourth embodiment.

As illustrated in FIG. 13, an image processing device 300 according to the fourth embodiment includes a computation unit 310 instead of the computation unit 140 illustrated in FIG. 5. The configurations and the operations of the respective components of the image processing device 300 other than the computation unit 310 are similar to those in the first embodiment.

The computation unit 310 includes a spectrum estimating unit 311 and an absorbance calculating unit 312 instead of the absorbance calculating unit 141 illustrated in FIG. 5.

The spectrum estimating unit 311 estimates the optical spectrum from an image based on image data read from the image data storage unit 132. More specifically, each of a plurality of pixels constituting an image is sequentially set to be a pixel to be estimated, and the estimated spectral transmittance T̂(x) at a point on an object corresponding to a point x on an image, which is the pixel to be estimated, is calculated from a matrix representation G(x) of the pixel value at the point x according to the following formula (26). The estimated spectral transmittance T̂(x) is a matrix having estimated transmittances t̂(x,λ) at respective wavelengths λ as elements. In addition, in the formula (26), a matrix W is an estimation operator used for Wiener estimation.

\hat{T}(x)=WG(x) (26)

The absorbance calculating unit 312 calculates absorbance at each wavelength λ from the estimated spectral transmittance T̂(x) calculated by the spectrum estimating unit 311. More specifically, the absorbance a(λ) at a wavelength λ is calculated by obtaining a logarithm of each of the estimated transmittances t̂(x,λ), which are elements of the estimated spectral transmittance T̂(x).

The operations of the component amount estimating unit 142 to the depth estimating unit 144 are similar to those in the first embodiment.

According to the fourth embodiment, estimation of a depth may also be performed on an image generated based on a broad signal value in the wavelength direction.

### Fifth Embodiment

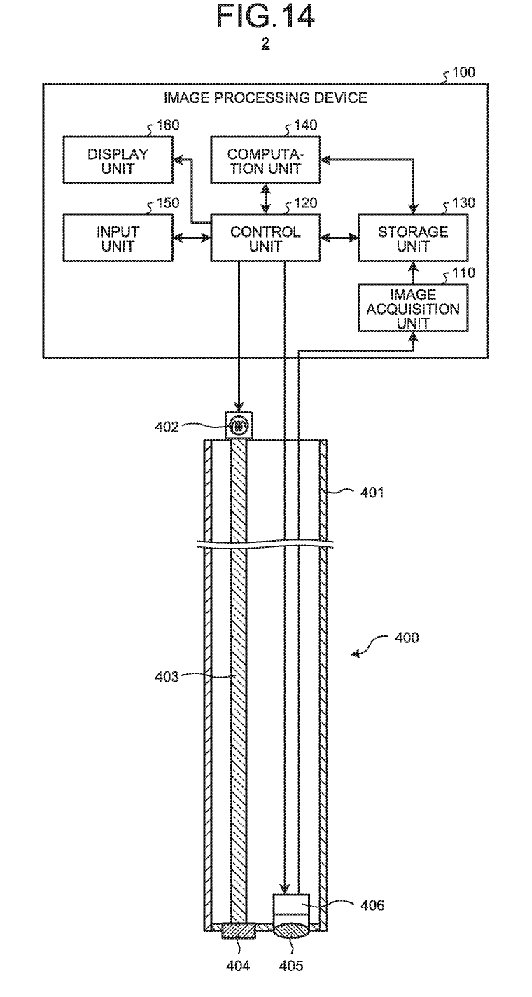

Next, a fifth embodiment is described. FIG. 14 is a schematic diagram illustrating an example configuration of an imaging system according to the fifth embodiment. As illustrated in FIG. 14, an endoscope system 2 that is an imaging system according to the fifth embodiment includes an image processing device 100, and an endoscope apparatus 400 for generating an image of the inside of a lumen by inserting a distal end into a lumen of a living body and performing imaging.

The image processing device 100 performs predetermined image processing on an image generated by the endoscope apparatus 400, and generally controls the whole endoscope system 2. Note that the image processing devices described in the second to fourth embodiments may be used instead of the image processing device 100.

The endoscope apparatus 400 is a rigid endoscope in which an insertion part 401 to be inserted into a body cavity has rigidity, and includes the insertion part 401, and an illumination part 402 for generating illumination light to be emitted to the object from the distal end of the insertion part 401. The endoscope apparatus 400 and the image processing device 100 are connected with each other via a cable assembly of a plurality of signal lines through which electrical signals are transmitted and received.

The insertion part 401 is provided with a light guide 403 for guiding illumination light generated by the illumination part 402 to the distal end portion of the insertion part 401, an illumination optical system 404 for irradiating an object with the illumination light guided by the light guide 403, an objective lens 405 that is an imaging optical system for forming an image with light reflected by an object, and an imaging unit 406 for converting light with which an image is formed by the objective lens 405 into an electrical signal.

The illumination part 402 generates illumination light of each of wavelength bands into which a visible light range is divided under the control of the control unit 120. Illumination light generated by the illumination part 402 is emitted by the illumination optical system 404 via the light guide 403, and an object is irradiated with the emitted illumination light.

The imaging unit 406 performs imaging operation at a predetermined frame rate, generates image data by converting light with which an image is formed by the objective lens 405 into an electrical signal, and outputs the electrical signal to the image acquisition unit 110, under the control of the control unit 120.

Note that a light source for emitting white light may be provided instead of the illumination part 402, a plurality of optical filters having different spectral characteristics may be provided at the distal end portion of the insertion part 401, and multiband imaging may be performed by irradiating an object with white light and receiving light reflected by the object through an optical filter.

While an example in which an endoscope apparatus for a living body is applied as the imaging device from which the image processing devices according to the first to fourth embodiments acquire an image has been described in the fifth embodiment, an industrial endoscope apparatus may be applied. In addition, a flexible endoscope in which an insertion part to be inserted into a body cavity is bendable may be applied as the endoscope apparatus. Alternatively, a capsule endoscope to be introduced into a living body for performing imaging while moving inside the living body may be applied as the endoscope apparatus.

### Sixth Embodiment

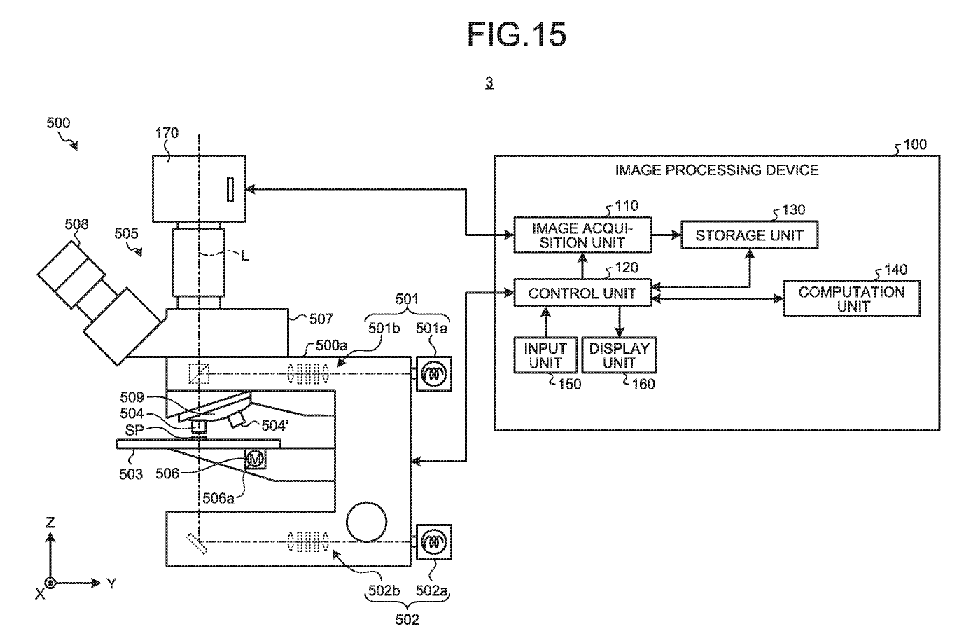

Next, a sixth embodiment will be described. FIG. 15 is a schematic diagram illustrating an example configuration of an imaging system according to the sixth embodiment. As illustrated in FIG. 15, a microscope system 3 that is an imaging system according to the sixth embodiment includes an image processing device 100, and a microscope apparatus 500 provided with an imaging device 170.

The imaging device 170 captures an object image enlarged by the microscope apparatus 500. The configuration of the imaging device 170 is not particularly limited, and an example of the configuration includes a monochromatic camera 171, a filter unit 172, and a tube lens 173 as illustrated in FIG. 6.

The image processing device 100 performs predetermined image processing on an image generated by the imaging device 170, and generally controls the whole microscope system 3. Note that the image processing devices described in the second to fifth embodiments may be used instead of the image processing device 100.

The microscope apparatus 500 has an arm 500a having substantially a C shape provided with an epi-illumination unit 501 and a transmitted-light illumination unit 502, a specimen stage 503 which is attached to the arm 500a and on which an object SP to be observed is placed, an objective lens 504 provided on one end side of a lens barrel 505 with a trinocular lens unit 507 therebetween to face the specimen stage 503, and a stage position changing unit 506 for moving the specimen stage 503.

The trinocular lens unit 507 separates light for observation of an object SP incident through the objective lens 504 to the imaging device 170 provided on the other end side of the lens barrel 505 and to an eyepiece unit 508, which will be described later. The eyepiece unit 508 is for a user to directly observe the object SP.

The epi-illumination unit 501 includes an epi-illumination light source 501a and an epi-illumination optical system 501b, and irradiates the object SP with epi-illumination light. The epi-illumination optical system 501b includes various optical members (a filter unit, a shutter, a field stop, an aperture diaphragm, etc.) for collecting illumination light emitted by the epi-illumination light source 501a and guiding the collected light toward an observation optical path L.

The transmitted-light illumination unit 502 includes a transmitted-light illumination light source 502a and a transmitted-light illumination optical system 502b, and irradiates the object SP with transmitted-light illumination light. The transmitted-light illumination optical system 502b includes various optical members (a filter unit, a shutter, a field stop, an aperture diaphragm, etc.) for collecting illumination light emitted by the transmitted-light illumination light source 502a and guiding the collected light toward the observation optical path L.

The objective lens 504 is attached to a revolver 509 capable of holding a plurality of objective lenses 504 and 504′, for example) having different magnification from each other. The imaging magnification may be changed in such a manner that the revolver 509 is rotated to switch between the objective lenses 504 and 504′ facing the specimen stage 503.

A zooming unit, including a plurality of zoom lenses and a drive unit for changing the positions of the zoom lenses, is provided inside the lens barrel 505 The zooming unit zooms in or out an object image within an imaging visual field by adjusting the positions of the zoom lenses.

The stage position changing unit 506 includes a drive unit 506a such as a stepping motor, and changes the imaging visual field by moving the position of the specimen stage 503 within an XY plane. In addition, the stage position changing unit 506 focuses the objective lens 504 on the object SP by moving the specimen stage 503 along a Z axis.

An enlarged image of the object SP generated by such a microscope apparatus 500 is subjected to multiband imaging by the imaging device 170, so that a color image of the object SP is displayed on the display unit 160.

The present disclosure is not limited to the first to sixth embodiments as described above, but the components disclosed in the first to sixth embodiments may be appropriately combined to achieve various disclosures. For example, some of the components disclosed in the first to sixth embodiments may be excluded. Alternatively, components presented in different embodiments may be appropriately combined.

According to the present disclosure, a plurality of component amounts are estimated by using a plurality of reference spectra at different depths of tissue for each of two or more kinds of light absorbing components contained respectively in two or more kinds of tissue including the specified tissue, and the depths of the tissue containing the light absorbing components are estimated based on the ratio of the component amounts estimated for each light absorbing component, which reduces the influence of light absorbing components other than that contained in the specified tissue and allows estimation of the depth at which the specified tissue is present with high accuracy even when two or more kinds of tissue is present in an object.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device adapted to estimating a depth of specified tissue included in an object based on an image obtained by capturing the object with light with wavelengths, the image processing device comprising:
an absorbance calculating unit configured to calculate absorbances at the wavelengths based on pixel values of pixels constituting the image;
a component amount estimating unit configured to estimate each of component amounts by using reference spectra at different depths of tissue for each of two or more kinds of light absorbing components contained respectively in two or more kinds of tissue including the specified tissue based on the absorbances;
a ratio calculating unit configured to calculate a ratio of component amounts estimated for a light absorbing component contained in at least the specified tissue; and
a depth estimating unit configured to estimate at least a depth of the specified tissue in the object based on the ratio.

2. The image processing device according to claim 1, wherein
the component amount estimating unit estimates a first component amount by using a reference spectrum at a first depth and estimates a second component amount by using a reference spectrum at a second depth deeper than the first depth for each of the two or more kinds of light absorbing components;
the ratio calculating unit calculates a ratio of either the first component amount or the second component amount to a sum of the first and second component amounts of a light absorbing component contained in at least the specified tissue; and
the depth estimating unit determines whether the specified tissue is present at a surface or at a depth of the object by comparing the ratio with a threshold.

3. The image processing device according to claim 1, wherein
the component amount estimating unit estimates a first component amount by using a reference spectrum at a first depth and estimates a second component amount by using a reference spectrum at a second depth deeper than the first depth for each of the two or more kinds of light absorbing components;
the ratio calculating unit calculates a ratio of the first component amount to the first and second component amounts of a light absorbing component contained in at least the specified tissue; and
the depth estimating unit estimates a depth of the specified tissue depending on a magnitude of the ratio.

4. The image processing device according to claim 1, wherein
the specified tissue is blood, and
a light absorbing component contained in the specified tissue is oxygenated hemoglobin.

5. The image processing device according to claim 1, further comprising:
a display unit configured to display the image; and
a control unit configured to determine a display mode for a region of the specified tissue in the image depending on a result of estimation by the depth estimating unit.

6. The image processing device according to claim 4, further comprising:
a second depth estimating unit configured to estimate a depth of tissue other than the specified tissue among the two or more kinds of tissue based on a result of estimation by the depth estimating unit.

7. The image processing device according to claim 6, wherein
the second depth estimating unit estimates that the tissue other than the specified tissue is present at a depth of the object when the depth estimating unit estimates that the specified tissue is present at a surface of the object, and
the second depth estimating unit estimates that the tissue other than the specified tissue is present at the surface of the object when the depth estimating unit estimates that the specified tissue is present at a depth of the object.

8. The image processing device according to claim 7, further comprising
a display unit configured to display the image; and
a display setting unit configured to set a display mode for a region of the tissue other than the specified tissue in the image depending on a result of estimation by the second depth estimating unit.

9. The image processing device according to claim 6, wherein the tissue other than the specified tissue is fat.

10. The image processing device according to claim 1, wherein the number of wavelengths is not smaller than the number of the light absorbing components.

11. The image processing device according to claim 1, further comprising:
a spectrum estimating unit configured to estimate an optical spectrum based on pixel values of pixels constituting the image, wherein
the absorbance calculating unit calculates the absorbances at the wavelengths based on the optical spectrum estimated by the spectrum estimating unit.

12. An imaging system comprising:
the image processing device according to claim 1;
an illumination part configured to generate illumination light with which the object is irradiated;
an illumination optical system configured to emit the illumination light generated by the illumination part to the object;
an imaging optical system configured to form an image with light reflected by the object; and
an imaging unit configured to convert the light with which an image is formed by the imaging optical system into an electrical signal.

13. The imaging system according to claim 12, comprising:
an endoscope provided with the illumination optical system, the imaging optical system, and the imaging unit.

14. The imaging system according to claim 12, comprising:
a microscope apparatus provided with the illumination optical system, the imaging optical system, and the imaging unit.

15. An image processing method for estimating a depth of specified tissue included in an object based on an image obtained by capturing the object with light with wavelengths, the image processing method comprising:
calculating absorbances at the wavelengths based on pixel values of pixels constituting the image;
estimating each of component amounts by using reference spectra at different depths of tissue for each of two or more kinds of light absorbing components contained respectively in two or more kinds of tissue including the specified tissue based on the absorbances;
calculating a ratio of component amounts estimated for a light absorbing component contained in at least the specified tissue; and
estimating at least a depth of the specified tissue in the object based on the ratio.

16. A non-transitory computer-readable recording medium with an executable program stored thereon, the program being adapted to estimating a depth of specified tissue included in an object based on an image obtained by capturing the object with light with wavelengths, and the program causing a processor to execute:
calculating absorbances at the wavelengths based on pixel values of pixels constituting the image;
estimating each of component amounts by using reference spectra at different depths of tissue for each of two or more kinds of light absorbing components contained respectively in two or more kinds of tissue including the specified tissue based on the absorbances;
calculating a ratio of component amounts estimated for a light absorbing component contained in at least the specified tissue; and
estimating at least a depth of the specified tissue in the object based on the ratio.
